(19) United States
(12) Patent Application Publication
(10) Pub. No.: US 2014/0296791 A1
(43) Pub. Date: Oct. 2, 2014

(54) PRE-FILLED MULTI-CHAMBER DEVICE FOR SEQUENTIAL DELIVERY

(71) Applicant: Season Sze-Shun Wong, College Station, TX (US)

(72) Inventor: Season Sze-Shun Wong, College Station, TX (US)

(21) Appl. No.: 14/191,438

(22) Filed: Feb. 27, 2014

Related U.S. Application Data
(60) Provisional application No. 61/850,969, filed on Feb. 27, 2013.

(51) Int. Cl. A61M 5/19

(52) U.S. Cl. CPC A61M 5/19; A61M 2005/1787
USPC 604/191

(57) ABSTRACT

The present invention relates to a pre-filled, syringe-like device to delivery multiple fluids (liquid and gas). Different segment of fluids are stored separated and then dispensed sequentially from the device when the plunger of the syringe is pressed. To enable the sequential delivery of pre-filled fluids, the invention includes a novel placement of connector hub(s) on the side of the barrel to allow it to function as intended. This allows fluids to be stored in a single body but in separate compartments and the fluids can be sequentially dispensed by a single plunging mechanism. This is in contrast to other dispensing methods where multiple steps are needed to sequentially dispense the fluids out of the container. In other invention, multiple barrels structures and manifold are also needed to perform sequential delivery. At the connectors, syringe needles, tubing, or a misting-delivering nozzle can be coupled to provide the user-desired dispensing patterns.

### CROSS-REFERENCE TO RELATED APPLICATIONS

This application claims the benefits of provisional patent application Ser. No. 61/850,969 titled PRE-FILLED MULTI-CHAMBER DEVICE FOR FLUID DELIVERY filed Feb. 27, 2013, by Season S. Wong (Wong Application).

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD OF INVENTION

This invention relates to unique placement of fluid outlets and use of conventional stoppers in single medical syringe-like barrel to provide multi-chambered sequential delivery fluids that include liquids and gases. This invention can be used to deliver drugs or deliver reagents to perform experimental steps.

### BACKGROUND AND DESCRIPTION OF RELATED ART

The present invention relates generally to a pre-filled syringe or cartridge and more particularly to a pre-filled syringe-like barrel capable of separated storage of two and more different fluids (liquid and gases) by implementing multiple stoppers inside a single syringe barrel-like structure. The separated fluids can be dispensed sequentially at a user-determined speed by a single plunging mechanism. This unique capability is achieved by placing additional fluids outlet(s) on the barrel body. These outlets are not found in conventional syringes and cartridges.

Conventional methods of dispensing different fluids require first storing the fluids in different containers. These containers need to be opened and then transported by using a device such as a pipette, vacuum, spoon, etc. Using the example of carrying a biological experiment where different liquids need to be added one by one, the conventional method will involve many steps of opening containers and transferring the fluids into a reaction chamber. Our invention allows all the necessary fluids to be stored in a single device prior to its use and these fluids to be dispensed sequentially using a single plunging mechanism. The pre-determined volume of the fluids stored in the barrel body is highly flexible and can be easily controlled by the spacing between the stoppers used in the barrel and the length of the barrel. The number of the fluids that can be stored is limited only by the number of stoppers used in the barrel to separate the fluids.

The apparatus we describe can be used in medical drug delivery as multiple chamber syringes. For example, it can deliver multiple, pre-measured, drugs via an intravenous (IV) tube, followed by flushing the IV tube with saline or water that are also prefilled in the same barrel. It can also be used to deliver any prefilled fluids desired by the users. For example, it can used to perform biological procedures that require precise amount and delivery order in a very simple manner. While multi-chamber syringes in various forms are well known, most multi-chamber syringes in the market are offered for use as mixing syringes and for sequential delivery of disparate fluids, maintaining the fluids separate until delivered. Almost all commercialized apparatus can only handle two components in practice. Mixing syringes most often provide features for mixing contents of the chambers and for delivering the mixed fluids simultaneously.

Conventionally, within each serial delivery syringe, chambers are separated by an intermediate sliding stopper which receives motive force communicated through an intermediate fluid from a primary stopper which is part of a plunger assembly against which an external force is applied. For the disparate fluids to be dispensed sequentially or serially, each intermediate stopper must provide a fluid tight seal until all fluid from a distal chamber is evacuated from the syringe. Once the syringe is so purged, that intermediate stopper must be breached or bypassed to permit dispensing of the contents of a proximal or intermediate chamber.

An example of a multi-chamber syringe is provided in U.S. Pat. No. 4,929,230 titled SYRINGE CONSTRUCTION and issued May 29, 1990 to Frederick W. Pfleger (Pfleger). Pfleger teaches a distortable piston which is used as the intermediate stopper. The piston of Pfleger collapses upon contact with a distal end of a syringe to provide a fluid pathway to dispense contents from the intermediate chamber. While a syringe made, as an example, according to Pfleger appears to provide a solution for sequentially dispensing disparate fluids, there are a series of concerns which would necessarily be associated with using such a syringe to dispense sequential doses of medications. For example, a deformable piston, having a hollow center, such as the stopper of Pfleger would not have zero dead space.

Other art, such as U.S. Pat. No. 6,027,481 issued Feb. 22, 2000 to Laurent Barrelle, et al. (Barrelle) and U.S. Pat. No. 5,851,200 issued Dec. 22, 1998 to Tetsure Higashikawa, et al. (Hagashikawa) disclosed multi-chamber syringes with sliding valves. However, in each case Barrelle and Higashikawa teach special structure imposed upon a syringe barrel (a channel in the case of Barrelle and a bulge in the case of Higashikawa) which is used to provide a fluid pathway about a stopper. Special stoppers are also needed in those inventions.

Another U.S. Pat. No. 6,723,074 B1, titled SEQUENTIAL DELIVERY SYRINGE and issued Apr. 20, 2004 to Thor R. Halseth (Halseth) teaches a sequential delivery syringe which utilizes a modification to a discharge opening of a syringe for providing access to a rear chamber of a two chamber syringe. The modification comprises affixing a piercing member at the discharge opening. The piercing member punctures a “mid-piston” and a collapsible bag disposed in a rear chamber to provide access to fluid in the bag. Access occurs when the mid-piston is displaced by action of a plunger and stopper piston to cause the mid-piston and bag to contact the piercing member.

Another U.S. Pat. No. 7,789,862, B2, titled Multi-chamber, sequential dose dispensing syringe and issued Dep. 7, 2010 to Gale H. Thorne (Thorne) teaches a valve assembly is disclosed which effectively partitions a syringe into proximal and distal chambers to provide a multi-chamber, sequentially dispensing syringe apparatus. Incorporated in the valve assembly is a valved stopper having a valve (which may be a slit valve), an impact sensor which opens the valve upon impact between the valve assembly and internal distal end of the syringe and a gas separator which separates liquid from gas disposed in the proximal chamber to assure gas is not delivered therefrom. The stopper is a much more complicated stopper than the conventional stoppers used in this invention. In addition, the number of the stoppers to be incorporated is limited.

### CROSS-REFERENCE TO RELATED APPLICATIONS

U.S. Patents which are related to the inventions disclosed herein.

| | | |
|---|---|---|
| US 20120323173 A1 | December 2012 | Thorne et al. |
| US 20120265171 A1 | October 2012 | Thorne et al. |
| U.S. Pat. No. 7,789,862 B2 | September 2010 | Thorne et al. |
| US20080114304 | May 2008 | Nalesso et al. |
| US 20040171984 A1 | September 2004 | Greenfield |
| U.S. Pat. No. 8,162,875 | April 2012 | Braga et al. |
| U.S. Pat. No. 7,998,106 B2 | August 2011 | Thorne, Jr. et al. |
| U.S. Pat. No. 7,217,253 B2 | May 2007 | Slate et al. |
| U.S. Pat. No. 7,101,354 B2 | September 2006 | Thorne, Jr. et al. |
| U.S. Pat. No. 7,048,720 B1 | May 2006 | Thorne, Jr. et al. |
| U.S. Pat. No. 6,997,910 B2 | February 2006 | Howlette, et al. |
| U.S. Pat. No. 6,723,074 B1 | April 2004 | Thor |
| U.S. Pat. No. 5,865,799 | February 1999 | Tanaka et al. |
| U.S. Pat. No. 5,298,024 | March 1994 | Frank |

These patents disclose backgrounds, uses and basic performance requirements of sequential dispensing or mixing syringes, respectively, and are hereby cited as references for such. While the above cited art, and other art generally related, provide a basis for commercial devices, there are some basic operational criteria which are not completely met by prior art. These criteria include:
1. True multi-chamber with a low-cost and user-friendly design. Most product can only deliver two components.
2. Scalable. Most existing technologies is limited to small volume syringes (3 mL and less). The current invention can lead to highly flexible diameter of barrel to be used. Syringe sizes from 1 mL to 60 mL have been reduced to practice by us via building prototype units.
3. Biocompatibility. Unlike other designs, where the stoppers have multiple parts and made with different raw materials, the stoppers we can use are simple to make and can be made with homogenous materials throughout the stopper. The barrel and stoppers in our convention can be fabricated easily using well-characterized and inert materials that are already commonly used in medical syringe production.

The invention disclosed herein, in appropriate embodiments, is unique in its ability to provide economical, highly reliable, robust, error-free and user-friendly ways of delivery multiple fluids sequentially.

### DEFINITION OF TERMS

Following is a brief list of clarifying definitions for terms used in this Application:
barrel n: a cylindrical elongated portion of a conventional syringe which is substantially of constant diameter along a long axis of the syringe, open on one end to receive a plunger tip and plunger rod assembly used for displacing fluid within the barrel and partially closed at an opposite end except for an orifice or portal through which fluid is ejected or aspirated. Our invention includes placement of additional outlets (or orifice or portal) along the cylindrical elongated barrel.
Stopper: a plug or a mass which is sufficiently large in cross section to fill and seal a hole
chamber n: a volumetric portion of a divided barrel
conventional adj: sanctioned by general custom; i.e. commonplace, ordinary
disparate n: when used to describe a first volume of contents relative to another volume of contents, the first volume of contents being kept distinctly separate from the other volume of contents.
distal adj: a term which depicts placement away from a reference point (e.g. away from a user of a syringe)
front adj/n: when referenced to a syringe, a distally disposed or a distally disposed site (e.g. a front of a syringe comprises the luer fitting orifice)
fluids n: a substance (e.g. a liquid or gas) which tends to take the shape of a container. In physics, a fluid is a substance that continually deforms (flows) under an applied shear stress. Fluids are a subset of the phases of matter and include liquids, gases, gel-like materials, plasmas and, to some extent, plastic solids.
gas n: a fluid which is neither solid nor liquid
liquid n: a fluid which is neither solid nor gaseous, free flowing like water.
plunger rod n: a portion of a syringe piston apparatus, usually affixed to a plunger tip, to which force is applied to displace fluid within a syringe-like barrel
plunger n: a portion of a syringe piston apparatus usually affixed to a plunger rod which is used to displace fluid within a syringe-like barrel
proximal adj: opposite of distal (e.g. a term which depicts placement nearer to a reference point)
rear adj: opposite from front (i.e. generally associated with a part of a syringe-like barrel which is proximal to a syringe user relative to an outflow orifice)
syringe n: a medical device used for injecting or withdrawing fluids
valve n: a device which has at least two stable states, in one state being closed to flow and in the other state being open to flow

### BRIEF SUMMARY AND OBJECTS OF THE INVENTION

A prefilled syringe-like device, according to the present invention, includes a tubular body having unique openings which would be closable by a stopper-plug and adapted to accept a fluidic connector or needle. Unlike conventional syringes where there is a small opening in one end and a larger opening to accept a plunger, our invention places additional opening(s) to the side of the tubular body. The tubular body's front end is still also closable by a plug at the connector hub and its rear, larger diameter end is closable by a plunger with a rod.

Inside the tubular body (barrel), movable partitions divide the interior space of the tubular body into multiple compartments in a fluid-sealing manner. The partitioning of the stopper-plug seals are independent of each other and their spacing is user-determined. The first compartment is the one closest to advancing towards the side opening and it stores the first fluid (liquid and gas). The second compartment stores a second fluid. Additional compartments can be created using additional stoppers. The stopper provides a liquid-tight environment. The compartments (stopper seals and the liquids stored inside), being slidable axially, move simultaneously when under pressure by the plunger. The action of the plunger on the stoppers and the side opening allows dispersing fluids sequentially via the side opening.

Unlike a conventional syringe barrel, the main opening (e.g., a needle hub) of the preferred embodiment is located on the side of the tubular body. Inside the barrel, movable rubber stoppers divide the interior space into multiple chambers in a liquid or air-tight manner. The volume and type of fluids being loaded are flexible. Additional chambers can be created using more stoppers. The preferred embodiment also allows the storage of air plugs. The chambers and the stoppers, being slidable axially, move simultaneously when under pressure by the plunger.

Immediately before use, the barrel's side and end openings should be turned to the open position from a closed position. When the plunger is pushed, all the stoppers and the materials inside the chambers then advance together. When the first stopper moves past the side opening, the pressure generated by the plunging force will push the fluid out through the opening. This occurs as long as the friction force between the stoppers and the barrel wall is bigger that the force needed to deliver the fluid out. In addition, the distal opening can be partially opened to ensure the plunger will deliver the fluid rather than advancing the stoppers too quickly. When this occurs, the first stopper stops moving while the rest continues to move axially. The fluid in the first chamber will be pushed out completely out when the second stopper makes contact with the first stopper. If the plunging force continues, the second stopper will pass over the opening and the fluid inside the second chamber will then exit the syringe via the side opening until the third stopper makes contact with the second stopper and effectively seals the outlet. The process can be repeated to deliver additional fluids.

Our preferred embodiment is a highly flexible design. Barrel of syringe sizes from 1 mL to 60 mL have been used to construct the sequential delivery device. The plunger can be operated by hand or by simple mechanical means such as a step-motor or syringe pump. There is no valve needed to control fluid delivery (as the stoppers function as valves). Our data confirms that by using hand or low-cost syringe pump operation, the setup can deliver flow rates as low as tens of μL/min to a few mL/min against pressure heads over 20 psi. An outlet channel as small as 300 micron in diameter has been used to deliver food-coloring fluids successfully into a steak-mimicking muscle. In addition to convenience, ease-of-use, and low-cost, the syringes and stoppers can be fabricated easily using well-characterized and inert materials already commonly used in medical syringe production.

The uniquely placed position of the side openings, the use of stoppers enable the sequential dispersion of fluids from the barrel without pushing the stoppers past the outlet too quickly. The outlet, when designed with luer-lock and luer-slip connectors, can be connected to downstream cartridges/syringes.

The advantages of the present invention will become apparent to those skilled in the art upon reading and understanding the following detailed description with reference to the accompanying figures.

### BRIEF DESCRIPTION OF THE DRAWINGS

This invention may be better understood, and its numerous objects and advantages will become apparent to those skilled in the art, by reference to the accompanying drawings,

FIG. 1 is a cross-sectional view through an embodiment of a syringe-like structure of the present invention. The outlet shown is connected to a stop-cock flow controller.

FIG. 2a to FIG. 2e show the general process of dispensing different fluids in sequence.

FIG. 3. A prospective view an embodiment of the device.

### DETAILED DESCRIPTION OF THE INVENTION

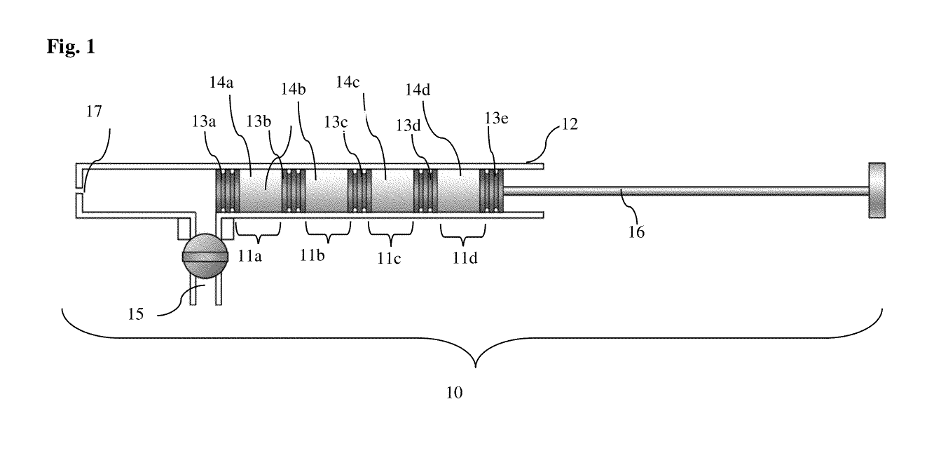

Referring now to the invention in more detail, FIG. 1 shows the basic components of the pre-filled device 10 which contains separate fluidic chambers (11) in a long and straight tubular structure 12. The tubular structure does not have to be firm and straight. The number of chambers should number at least two, though four chambers (11a, 11b, 11c, and 11d) are shown in the illustration as an example. There is no upper limit of number of chambers. Each chamber is separated by stoppers (13a to 13e) that seal its content from one another. The fluids stored in each chamber (14a, 14b, 14c, and 14d) can be the same or different. The stopper 13a to 13e used in the partitioning are similar to those used in syringe plunger seals. At the long straight side of the tubular structure 12 consists of an outlet opening 15 for a connector that can be capped or connected to tubing or a multi-way stopper (2-way and more) leading fluids away from the apparatus. At the proximal end of the tubular structure 12 includes a plunger 16 that has a seal that is made with materials similar to the stopper. At the other round (distal) end of the tubular structure 12 is an opening outlet 17.

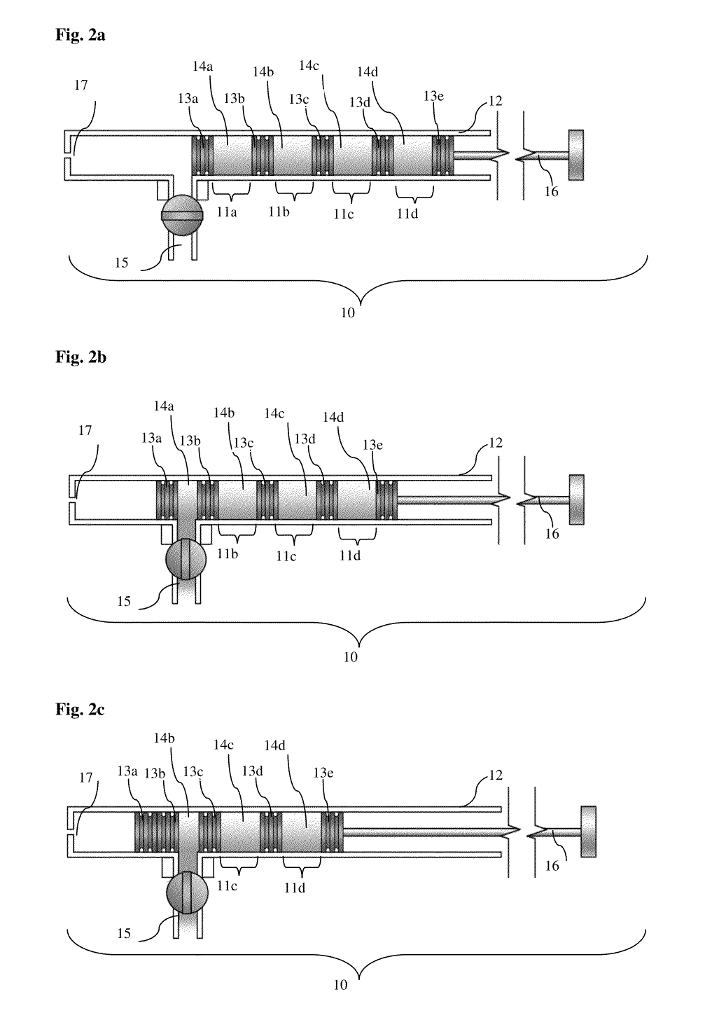

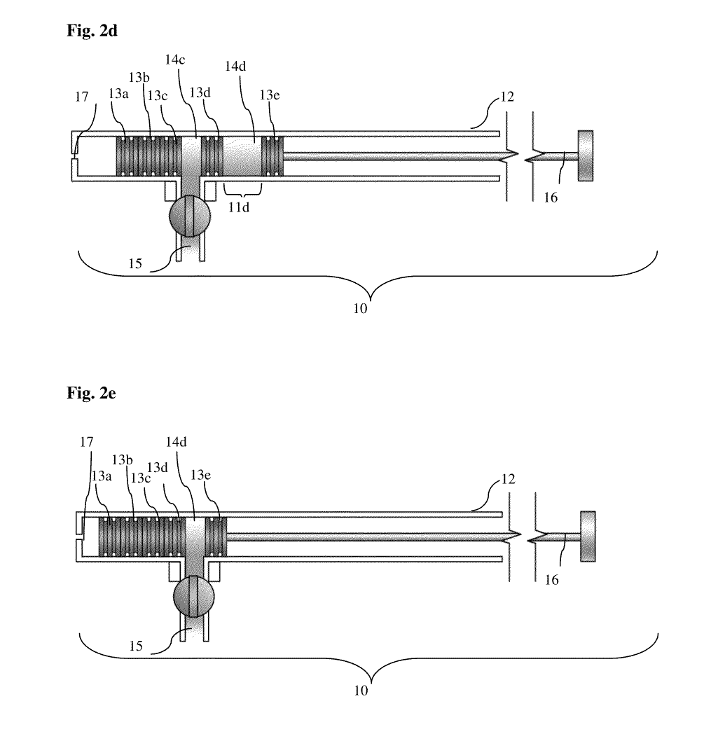

FIG. 2a to FIG. 2e shows the principle of how to operate the apparatus with four fluidic chambers. The full length of the plunger rod is not shown in the figure. The side opening 15 and the end opening 17 should be in an open position when the plunger 16 is pushed. When the plunger 16 is pushed, all the stopper-plug seals 13a to 13e and the fluids 14a to 14d inside the chambers move towards the distal end of the barrel structure with the end opening 17 and side outlet opening 15 (FIG. 2a). When the first stopper-plug seal 13a reaches the outlet 15, the pressure generated by the plunging force will push the fluid (14a) out through the outlet's opening 15 (FIG. 2b). After this occurs, the stopper 13a will stop moving while the rest of the stoppers (13b to 13e) will continue to move axially due to the action of the plunger 16. The fluid (14a) in the first chamber 11a will be pushed out when the second stopper 13b touches the first stopper 13a. If the plunging force continues, the second stopper 13b will pass over the side outlet opening 15 and the fluid (14b) inside the chamber 11b will start to be pushed out (FIG. 2c). When this occurs, the stopper-plug seal 13b will stop moving while the rest of the other stoppers (13c to 13e) and the fluids within (14b to 14d) will continue to move axially due to the action of the plunger 16. The fluid (14b) in the second chamber 11b will be emptied out when the third stopper-plug seal 13c touches the second stopper 13a (FIG. 2d). If the plunger continues to being pushed, similar to the previous steps, the fluids (14c and then 14d) in chambers 11c and 11d will be removed from the barrel at the end (FIG. 2e).

The unique position of the side outlet opening 15, the use of stopper 13a-13d, and the plunger 16 enables the sequential delivery of fluids from the barrel body. The plunger can be stopped at any moment at an user-controlled position (just stop pushing the plunger). The process can by again pushing the plunger. The unit can be operated by hand or by simple mechanical means such as a step-motor. The shape and dimension of the tubular structure 12 and each fluidic chamber 11 for the fluids are not limited to a circular tube. The cross-section of the chambers can be circular, square, hexagonal, etc as long as a complementarily shaped stoppers are made to separate the fluids.

In further detail, still referring to the invention of FIG. 1, the device's components of the device can be made of different materials so long as they can function as a leak-proof, pre-filled device to dispense fluids. The main body can be made from plastic (for example, but not limited to, polypropylene polyethylene, polycarbonate, and polystyrene), glass and any other suitable materials. The stopper-13 can be made from semi-soft materials such as plastics and polymers so long as they provide fluidic sealing and are resistant to the chemical and biological agents used in the fluids being stored.

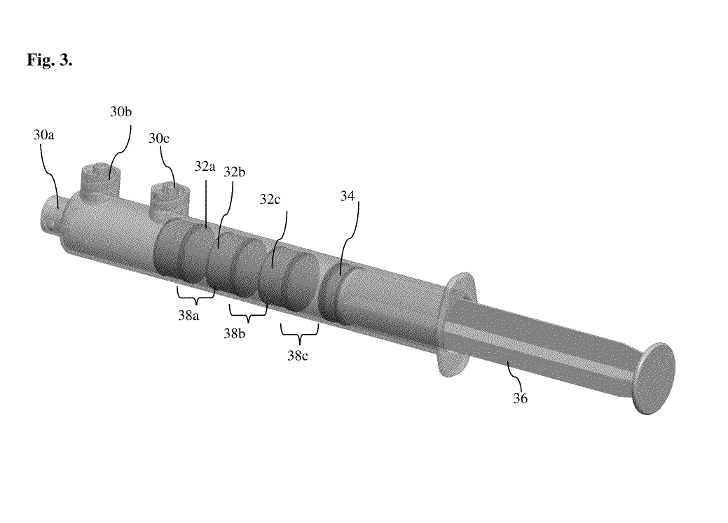

FIG. 3 shows the prospective way of a drawing we created for the fabrication of an injection mold. The three outlets are to be made as luer-lock connectors 30a, 30b, and 30c. Stoppers 32a, 32b, 32c, and the stopper (34) on the plunger rod 36 created three chambers 38a to 38c.

### ADVANTAGES OF THE INVENTION

Thus, the invention described herein has the advantages of (1) providing a prefilled syringe-like structure which can dispense multiple fluids sequentially using a single plunger without making mistake in the sequence of dispensing. (2) the volume of the fluids are pre-determined and pre-filled so that measurement of fluids is not needed immediately before dispensing. (3) A fluid needs to be emptied before that next fluid can be dispensed so the pre-measured volume of fluids will be dispended not not left in the device. (3) the device provides optimum conditions for carrying out complicated fluid dispensing tasks with an innovative but low-cost approach.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

What is claimed:

1. An apparatus that stores and dispenses multiple, pre-filled fluids sequentially in a syringe-like barrel in a controllable manner with a single plunger. The barrel comprising a hollow, elongated internal cylindrical surface which is contiguous to an open proximal end and to a distal end having a closed interior about an orifice through which fluid is vented or transferred. In addition, the barrel also contains one or more side outlets on the cylindrical surface where fluids can also be dispensed from the outlet when it is not closed by a cap or by the stopper inside the barrel. Stoppers are placed inside the barrels. Fluids are loaded in the barrels and separated by the stoppers. The plunger and plunger rod actuated by hand or any mechanical means pushes the stoppers and their contents in between along the barrel. Fluid is delivered through the opened side outlet when the stopper passes over the outlet and the pressure from the plunger forces the fluid out of the barrel rather than keep advancing the stoppers and their contents along the barrel, said apparatus comprising:
said syringe-like barrel having a hollow cylindrical wall having an internal surface which is concentrically disposed about an elongated axis, said the barrel surface comprising an open proximal end, one and more side end (outlets) and a distal end with both the distal and side ends having orifices through which fluid is transferred;
said side outlet along the cylindrical barrel and is located between proximal end and to a distal end. Said side outlet placed along the elongated axis of the barrel where fluids can be delivered out when actuated by the plunger and when stoppers inside the barrel are not blocking the outlets. The friction required to move the stoppers axially should be larger than the force need to push the fluid out of the outlet;
said side outlet that have a larger diameter orifice than the distal open end to ensure that the plunger force will deliver the fluids out via the said side outlet rather than moving the stoppers and the contents in between too quickly without deliver its content;
said stoppers for disposal within the barrel for dividing the barrel into a proximally disposed chamber, a distally disposed chamber, and additional chambers in between, stoppers being cylindrically shaped and sized to be slideably displaced along the surface and also function as plunger to push fluids while being so displaced;
said stopper diameter that matches that of the inside diameter of the barrel so that liquid and air-tight segments are generated but still easy enough for the plunger rod to move them;
said stoppers placed in the barrel that functions both as plugs between the fluids that seal them from interacting with one another and valves to keep the fluids from exiting the barrel via the side outlet when stoppers sit directly at the outlet;
The friction required to move the stoppers axially is larger than the force need to push the fluid out of the outlet. Therefore, when the valve is open, the plunger force is used to deliver fluid out of the barrel and not to advance the stoppers' positions inside the barrel;
said stoppers that have same diameter and match the barrel's inside diameter but the stoppers' thickness can be the same or different;
said chambers that generated by the spaced out stoppers inside the barrel can be filled with either liquid, gases, or a mixture of liquid and gases;
said plunger rod and plunger tip combination disposed to be displaced within said barrel by application of a directional force against said plunger rod for dislocating fluid thereby;
said side outlet is normally in a closed position when a stopper sits at a location inside the barrel that blocks the outlet opening and keeps its content from leaving the outlet;
said side outlet is normally in an open position when a stopper sits at a location inside the barrel that does not block the outlet;
said side outlet can be designed to be, but not limited as, a simple hole, a luer-lock connector design, a slip-lock connector design, a misting-delivering nozzle, a stop-cock connector, or tubing;
said distance between the distal end and the side outlet should measure longer than the combined thickness of all the stoppers placed inside the barrel;
said diameter of the barrel, the length of the barrel, and the number of stoppers placed inside the barrel to create chambers of fluids are unlimited;
said chemical composition and the viscosity of the fluids placed inside the barrel are unlimited;
said barrel can function when placed in any direction (vertically, horizontally, and any orientation in between);
said example materials used to make the apparatus (barrel, stoppers, plunger, plunger rod, outlets) can be, but not limited to, to glass, plastics, polymers, metal, etc.
